(19) United States
(12) Patent Application Publication
(10) Pub. No.: US 2011/0264148 A1
(43) Pub. Date: Oct. 27, 2011

(54) ORTHOPEDIC IMPLANT IN THE FORM OF A PLATE TO BE FIXED BETWEEN TWO BONE PARTS

(76) Inventors: Bernard Prandi, Rennes (FR); Marino Delmi, Conches (CH); J. Chris Coetzee, Eagan, MN (US); Lowell Well, Lake Forest, IL (US); Keith Wapner, Philadelphia, PA (US); Christopher P. Chiodo, Jamaica Plain, MA (US); Michael Coughlin, Boise, ID (US); Yves Tourne, Gieres (FR); Christophe Richard Walker, Loirral (GB); Donald R. Bohay, Ada, MI (US); John G. Anderson, Lowell, MI (US)

(21) Appl. No.: 12/918,116

(22) PCT Filed: Oct. 2, 2009
(86) PCT No.: PCT/FR2009/051878

§ 371 Date: Nov. 1, 2010

(30) Foreign Application Priority Data
Oct. 2, 2008 (FR) 0856693

(51) Int. Cl. A61B 17/80

(52) U.S. Cl. 606/286

(57) ABSTRACT

The plate is fixed by screws (3) engaged in holes (1a) formed in the thickness of the plate which is designed in such a way as to ensure an adjustable compression effort, created by a screwing effect, which brings the two bone parts closer together. The design consists of at least one recess (1b) having a cross-section defining a plurality of separate areas (1b1), (1b2) and (1b3) that can co-operate with a screw (3) in order to generate a progressive translational movement of the screw (3) during the screwing action, corresponding to a movement of at least one of the bone parts, and causing the compression effort. Said plate (1) comprises a recess (1c) for the introduction of a spindle (4) on the side of one of the bone parts, and another recess (1d) for the introduction of a spindle (5) on the side of the other bone part, one of said recesses (1c) consisting of a circular hole having a diameter which essentially corresponds to that of the spindle (4), and the other recess (1d) consisting of an oblong hole.

The invention relates to the technical field of orthopedic implants.

More particularly, the invention relates to a plate for arthrodesis or osteosynthesis adapted to be fixed between two bone parts.

In a manner known to one having ordinary skill in the art, this type of plate generally has holes for anchor screws, allowing arthrodesis between two bones or an osteosynthesis between two bone fragments. This is, for example, the case for the bones of the hand or foot, without however excluding other applications, particularly in the field of the spine. Depending on the pathological case to be treated, these plates can have a general rectilinear shape or have other geometric shapes.

From this state of the art, one of the objects the invention proposes to attain is to improve, in a sure and efficient manner, the compression in a precise direction between the bone parts held by the plate.

To attain this object, an orthopedic implant has been invented and designed in the form of a plate adapted to be fixed between two bone parts by screws engaged in throughgoing holes extending through the entire thickness of the plate.

According to the invention, the formations of the plate adapted to ensure, under a screwing effect, an adjustable compression force to bring the two bone parts closer together, are formed by at least one seat having a cross-section defining several distinct regions adapted to cooperate with a screw in order to generate a progressive straight-line displacement of the screw during the screwing action corresponding to a displacement of at least one of the bone parts, causing the compression force.

To attain the given object of obtaining a controlled and adjustable compression, one of the regions is constituted of a hole having a diameter D1 adapted to receive the screw head, whereas another region is a hole having a diameter D2 less than the diameter D1, and having, over a determined height, a slope between 40° and 60°, the regions being connected by an intermediate region having a slope in the direction of the region constituted by the hole having the diameter D1.

Advantageously, the slope of the intermediate region is comprised between 15° and 30°.

Another problem that the invention proposes to resolve is to ensure a temporary fixation of the plate so as to facilitate the fixation of the latter by the operator by means of pins and to allow, after the pins are set in place in one of the bone parts, sliding under the plate at the time of screwing while ensuring a compression according to a precise direction.

To solve such a problem, the plate has, in a known manner, a guide hole for the insertion of a pin on the side of one of the bone parts, and another guide hole for the insertion of a pin on the side of the other bone part.

According to the invention, one of the guide holes is a circular hole whose diameter corresponds substantially to that of the pin, and the other guide hole is an elongated slot.

To solve the given problem of respecting the anatomy, particularly in the case of an MTP arthrodesis plate, the axis of the elongated slot extends relative to the longitudinal axis of the plate at an angle between about 1° and 10°.

Generally, the plate has smooth and/or threaded holes adapted to receive anchor screws to the bone parts.

According to this base-design of the plate and of the pathological case to be treated:

Either the different holes are aligned;

Or some of the holes are arranged according to the apex of a triangle or of a quadrilateral.

In one embodiment, the plate is longitudinally centered so as to adapt to the curvature of the bone parts.

The invention is described hereinafter in more detail is with reference to the attached drawings, in which:

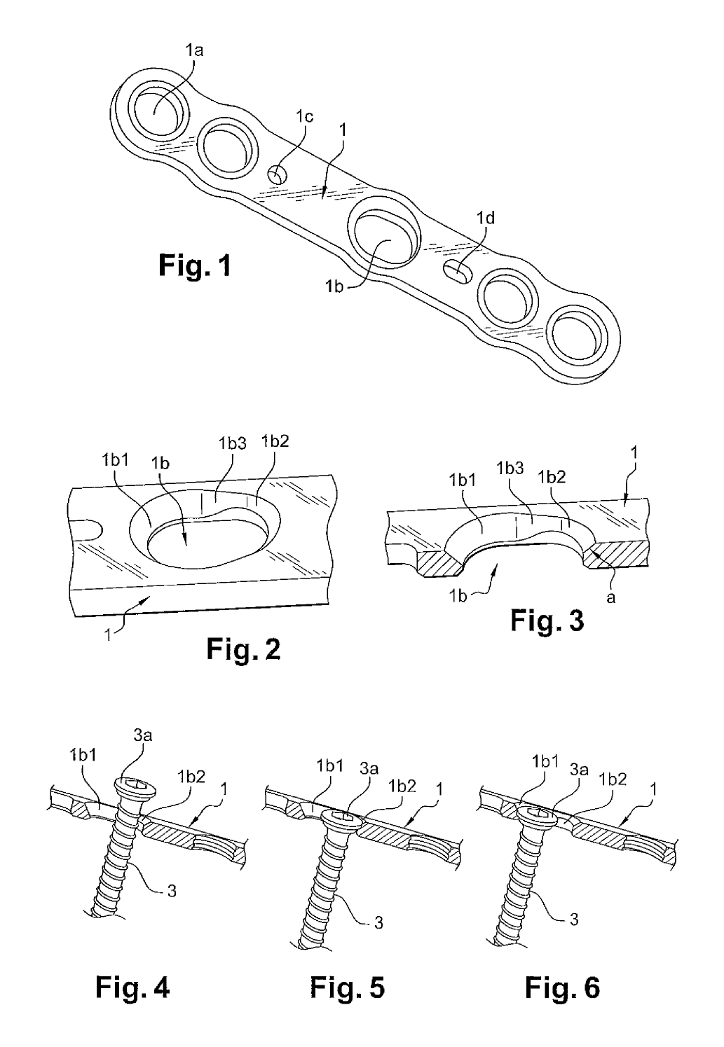

FIG. 1 is a perspective view of an embodiment of a plate according to the invention;

FIG. 2 is a partial, large-scale, perspective view showing the compression seat;

FIG. 3 is a longitudinal cross-section corresponding to FIG. 2;

FIGS. 4, 5, and 6 are partial, cross-sectional, schematic, perspective views showing the compression obtained as the screw is pressed into the compression seat;

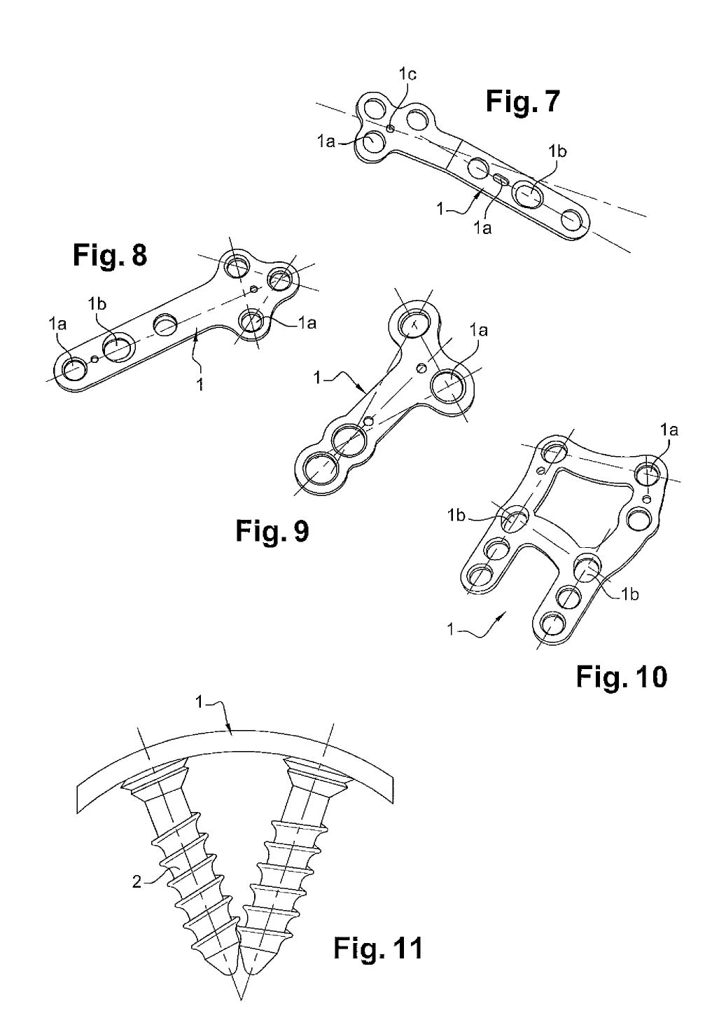

FIG. 7 is a perspective view of another embodiment of the plate, particularly for MTP arthrodesis;

FIGS. 8, 9, and 10 are perspective views of embodiments of plates of other shapes;

FIG. 11 shows an embodiment of a plate longitudinally shaped to fit to the curvature of the bone;

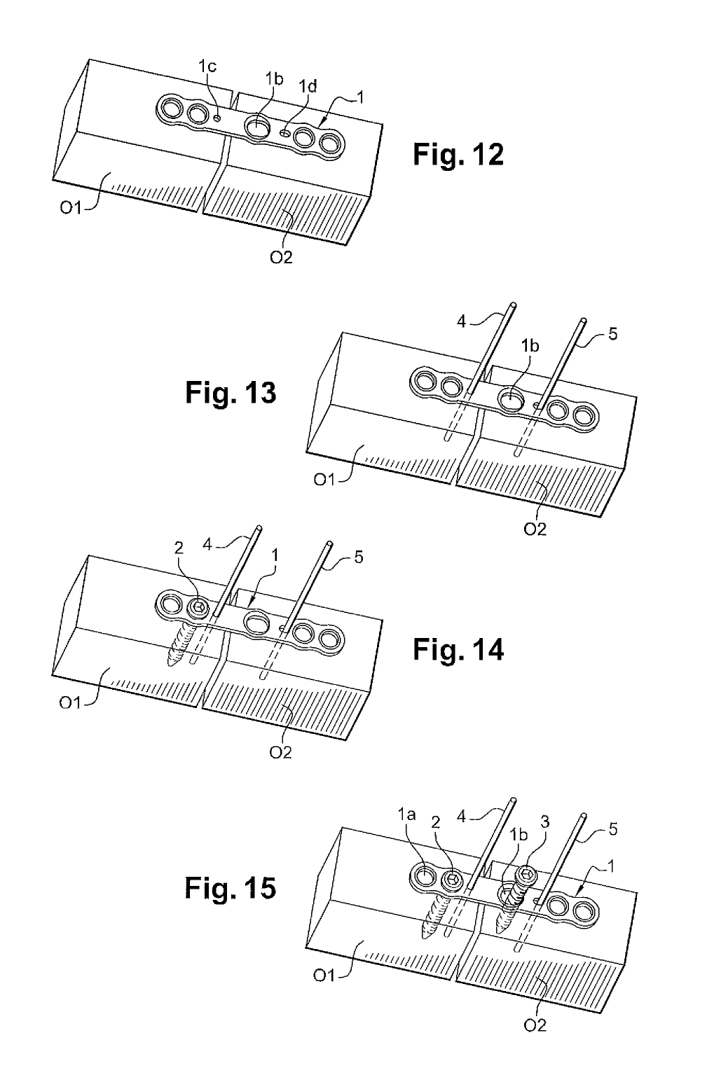

FIGS. 12 to 18 are perspective views showing the installation and fixation of the plate on two bone parts for compressing same together, the bone parts being schematically shown as parallepipedal blocks;

FIG. 19 is a partial view of the plate showing, schematically, two extreme positions of the initial boring to obtain maximum compression (position 1) or no compression (position 0).

The osteosynthesis and/or arthrodesis plate is indicated generally at 1. In a known manner, this plate 1 has smooth and/or threaded holes 1a for anchor screws 2 screwed into bone parts O1 and O2, as described below. According to a characteristic of the invention, the plate 1 has at least one compression seat or formation 1b.

As shown, in particular in FIGS. 2 and 3, this seat 1b has several distinct regions 1b1, 1b2, and 1b3 that allow the desired compression to be set, particularly as a function of the bone quality and as a function of the start position of a screw 3 in the seat.

More particularly, the seat 1b is formed by three distinct regions having the following characteristics:

The region 1b1 is a hole having a diameter D1, adapted to receive the head 3a of the screw 3.

The region 1b2 is a hole having a diameter D2 less than the diameter D1 and having, over a determined height, comprised between about 1 and 2 mm, a slope on the order of 40° to 60° relative to a base plane of the plate.

The regions 1b1 and 1b2 are connected by an intermediate transition region 1b3 having a down slope, that is, angled downward in the direction of the region 1b1. This slope is between about 15° and 30°.

Considering these characteristics, compressing together the two bone parts 01 and 02 is carried out as follows, with reference, in particular, to the FIGS. 4, 5, 6, 15, and 16.

A pilot hole is first made in one of the bone parts being considered according to the region 1b2 or 1b1 as a function of the compression one wants to obtain.

The screw 3 is inserted into the hole until its head 3a contacts the plate 1 (FIGS. 4 and 5).

Since the hole of the region 1b2 has a diameter smaller than that of the screw head 3, this head immediately slides so as to be offset from the axis of the region 1bi, causing a first, rapid compression.

With continued screwing action, the screw head 3a is slides along the slope of the intermediate region 1b3 causing a second compression until it reaches a stable position fitting in the hole 1b1 whose diameter is the same as that of the screw head 3a. Naturally, it is possible to stop at any intermediate position in order to precisely adjust the compression.

It must be noted also that the extent of compression generated is on the order of 0 to 3 mm and can be controlled as a function of the position of the initial passage. Therefore, it is possible to obtain maximum compression at a seat in position 1 and no compression at a seat in a position 0 (FIG. 19).

According to another characteristic, the plate 1 has at least one guide hole 1c for the insertion of a guide pin 4 for temporarily fixing the plate 1. Advantageously, the plate 1 has a guide hole 1c for the insertion of a pin 3 on the side of one of the bone parts O1 and another guide hole ld for the insertion of another guide pin 5 on the side of the other bone part O2.

Considering the effect of the desired compression, such as previously mentioned, the guide hole 1c is a circular hole whose diameter corresponds substantially to that of the pin 4, whereas the other guide hole 1d is an elongated slot, located on the side of the compression seat 1b.

These provisions therefore enable the bone to slide relative to the plate 1 at the time of screwing, while ensuring compression in a precise direction generally along the axis of the plate. The pins 4 and 5 are of any known and appropriate type, and perfectly know to one having ordinary skill in the art.

The elongated slot 1d for temporary fixation is particularly well adapted in the case of an MTP arthrodesis plate in order to take into account the anatomy according to which the angle between the phalange P1 and the metatarsus 1 is on the order of 10°. Indeed, a plate according to the state of the art makes it difficult to ensure the compression while keeping the above-described angle. According to the invention, the slot ld only has to be angularly oriented relative to the median axis of the plate 1 according to an angle of about 5° (FIG. 7).

As shown in the figures of the drawings, the plate 1 can have different shapes, with the holes 1a aligned as in FIG. 1 or arranged, all or in part on the corners of a triangle or of a quadrilateral (FIGS. 8, 9, and 10). More particularly, in FIG. 8, the plate is adapted to be used in the case of a Lapidus arthrodesis, in FIG. 9, for a basal osteotomy of the first metatarsus and for a ⅔ Lisfranc arthrodesis (FIG. 10). These provisions of the screw, in a triangle or in a quadrilateral, improve the mounting stability.

It must be noted also that the plate 1, no matter its geometric shape, can be bent longitudinally so as to adapt to the curvature of the bone (FIG. 6) making it possible, as a consequence, for the screws 2 to form an angle relative to one another (FIG. 11).

With reference to FIGS. 12 to 18 that show the setting in place of the plate according to the invention:

After the osteotomies have been carried out, the plate 1 is positioned between the two bone parts O1 and O2 (FIG. 12).

The surgeon stabilizes the plate 1 with the two temporary pins 4 and 5, one of which is engaged in the hole 1c and whereas the other one in the slot 1d (FIG. 13).

At least one screw 2 is engaged through the hole 1a and screwed into the bone part O1 where there is no compression formation 1b is not positioned (FIGS. 14).

The surgeon then screws the screw 3 in the compression lug 1b, choosing the extent of compression as previously indicated (FIG. 15).

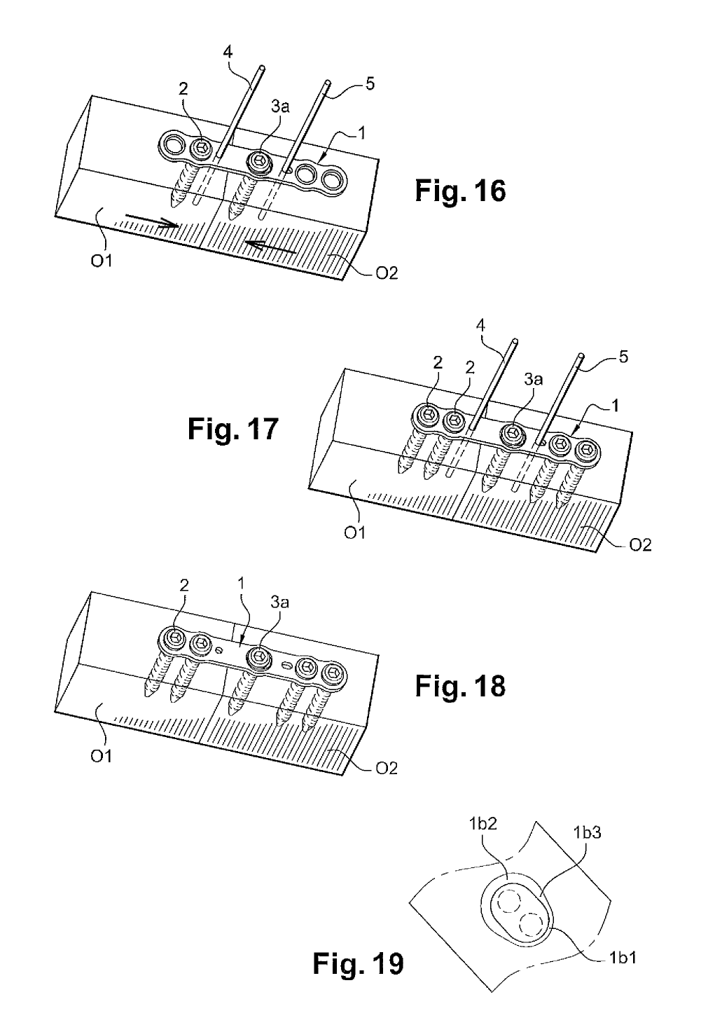

Once the compression is done (FIG. 16), the surgeon can screw in one or more additional screws 2 (FIG. 17), then remove the guide pins 4 and 5 (FIG. 18).

The advantages are readily apparent from the description.

1. An orthopedic implant in the form of a plate adapted to be fixed between two bone parts by screws engaged in throughgoing screw holes one of which has formations that ensure, when the respective screw is rotated, an adjustable compression force adapted to bring the two bone parts closer, wherein the formations are constituted of at least one seat having a shape seen in section delimiting several distinct regions adapted to cooperate with the respective screw in order to cause a progressive, straight-line displacement of the plate on tightening of the screw in the one screw hole and causing a displacement of at least one of the bone parts the plate having a guide hole for the insertion of a pin on the side of one of the bone parts and another guide hole for the insertion of a pin on the side of the other bone part, one of the guide holes being a circular hole whose diameter corresponds substantially to that of the respective pin and the other guide hole being an elongated slot.

2. The implant according to claim 1, wherein one of the regions is a hole having a diameter D1 adapted to receive the head of the screw, another region is constituted of a hole having a diameter D2 that is less than the diameter D1, and having, over a determined height, a slope of between 40° and 60° relative to a base plane of the plate, the regions being connected by an intermediate region having a slope angled down toward the region constituted of the hole having the diameter D1.

3. The implant according to claim 1, wherein the slope of the intermediate region is between 15° and 30°.

4. The implant according to claim 1, wherein an axis of the elongated slot is angularly oriented relative to a longitudinal axis of the plate at an angle between about 1° and 15°.

5. The implant according to one of claims 1 wherein the plate has smooth or threaded holes adapted to receive anchor screws to the bone parts.

6. The implant according to claim wherein the holes are aligned.

7. The implant according to claim 5, wherein some of the holes are arrayed at the corners of a triangle or of a quadrilateral.

8. The implant according to claim 1, wherein the plate is longitudinally bent so as to adapt to the curvature of the bone parts.

9. An orthopedic implant assembly for joining a pair of bone parts, the implant comprising:
an elongated plate extending at least generally in a plane and adapted to lie against both of the bone parts, the plate being formed with two throughgoing screw holes each aligned with a respective one of the bone parts and with two guide holes each also aligned with a respective one of the bone parts, one of the guide holes being circular and the other of the guide holes being a slot elongated generally parallel to the plate;
a pair of circular-section guide pins engageable through the guide holes, one of the pins fitting snugly in the circular guide hole and the other of the pins being slidable along the slot;
respective screws with heads engageable through the screw holes with the respective bone parts, one of the screw holes having a formation shaped to cam the plate and shift it axially relative to the respective bone part as the respective screw head presses against the formation.

10. The orthopedic implant assembly defined in claim 9 wherein the one screw holes is elongated generally parallel to the plate and has:
an outer end region in which the screw head is seatable and that has an edge relatively closely spaced from the respective bone part;
an inner end region having an edge engageable with the respective screw head, spaced further from the respective bone part than the edge of the outer end region and angled down toward the to respective bone part and outer end region; and
an angled intermediate region connecting the inner and outer end regions.

11. The orthopedic implant assembly defined in claim 10 wherein the inner end region extends at an angle of 40° to 60° to the plane of the plate.

12. The orthopedic implant assembly defined in claim wherein the intermediate region extends at an angle of 15° to 30° to the plane of the plate.
